(19) United States
(12) Patent Application Publication
(10) Pub. No.: US 2023/0329904 A1
(43) Pub. Date: Oct. 19, 2023

(54) Catheter System for Surgical Access and Therapeutics Delivery

(71) Applicant: THE BOARD OF TRUSTEES OF THE UNIVERSITY OF ILLINOIS, Urbana, IL (US)

(72) Inventors: Anthony E. Felder, Urbana, IL (US); Matt Tomback, Urbana, IL (US); Aaron Chicano, Urbana, IL (US); Yannek I. Leiderman, Chicago, IL (US); Kimberlee Wilkens, Urbana, IL (US); Peter Pfanner, Urbana, IL (US); Manuela Burek, Urbana, IL (US)

(21) Appl. No.: 17/777,528

(22) PCT Filed: Nov. 17, 2020
(86) PCT No.: PCT/US2020/060898

§ 371 Date: May 17, 2022

Related U.S. Application Data
(60) Provisional application No. 62/937,726, filed on Nov. 19, 2019.

(51) Int. Cl. A61F 9/00; A61F 9/007

(52) U.S. Cl. CPC A61F 9/0017; A61F 9/007; A61B 17/3423

(57) ABSTRACT

A catheter includes a bifurcated shaft body and a tip coupled to the bifurcated shaft body. The tip is configured to dissect a portion of a tissue in a controlled navigation for targeted delivery of a payload to an area of or within the tissue. In addition, the tip is adapted to bend along a shape of the area of the tissue.

### CROSS REFERENCE TO RELATED APPLICATIONS

This patent application claims priority to U.S. Provisional Patent Application No. 62/937,726 filed Nov. 19, 2019, entitled “Catheter System for Surgical Access/Therapeutics Delivery,” the entire disclosure of which is hereby incorporated by reference.

### FIELD OF DISCLOSURE

The present disclosure relates generally to catheter systems and, more specifically, to a steerable catheter system configured to dissect a portion of a tissue in a controlled navigation for targeted delivery of therapeutics.

### BACKGROUND

Diseases of the posterior segment of the eye are common causes of blindness and can be difficult to treat due to their location. Recently, there has been increased interest in the use of the suprachoroidal space (SCS) to deliver therapeutics to the posterior segment. This space is accessible through a trans-scleral approach and blunt dissection of the adjacent scleral and choroidal tissues. However, despite recent commercial interest, there are few tools designed specifically to provide access or targeted delivery of therapeutics to a localized region in the body, such as the posterior eye, subretinal space, and SCS that address the delicate anatomy of this or other surgical regions in the body.

Specifically, diseases of the retina, such as age-related macular degeneration and diabetic retinopathy, are common causes of blindness. While the pathophysiology of these diseases varies, their location in the posterior segment has historically made drug delivery challenging. These diseases, or stages thereof, are commonly treated by repeated intravitreal injections. However, this method precludes the targeted delivery of certain therapies, such as retinal implants, extracellular scaffolds, adeno-associated viral vectors, cellular transplants, and other genetic therapies, due to restrictions on size or location of the injected payload. Another potential delivery medium to the posterior segment is through the suprachoroidal space (SCS). The SCS spans the perimeter of the ocular globe and can be accessed via a trans-scleral approach with blunt dissection of the neighboring chorioretinal and scleral tissues. The SCS has been utilized for many purposes, including drug delivery, retention drainage of the anterior chamber, drainage of choroidal effusions, and repair of retinal detachment. Moreover, drug delivery within the SCS can increase the efficacy and bioavailability of the administered therapeutic, potentially reducing adverse side effects caused by delivery to adjacent tissues. Recently, there has been some commercial interest in developing tools or systems for the delivery of therapeutics to the SCS. For example, Clearside Biomedical has developed a microneedle for direct injection of agents into the SCS. While this methodology has demonstrated enhanced pharmacokinetics compared to traditional intravitreal injection, it cannot target localized regions of the SCS for delivery. Another system, the iTrack Microcatheter by iScience, was developed to navigate the SCS and deliver therapeutics, as demonstrated in minipigs. However, this system does not provide steerability of the catheter, making navigation through the SCS laborious, recursive, and unpredictable.

Moreover, current use of catheters in the SCS is limited. Previous studies used existing blunt- or olive-tipped catheters, as depicted in FIGS. 1A-1B and FIG. 1C-1D, respectively. Specifically, FIGS. 1A and 1B depict a portion of a catheter 10 having a blunt-shaped tip 12, and FIGS. 10 and 1D depict a portion of a catheter 14 having an olive-shaped tip 16. However, such known systems fail to provide a catheter or drug delivery system designed specifically for blunt dissection of the delicate extravascular tissues defining SCS.

### SUMMARY

In accordance with the principles of the present disclosure, an ophthalmic catheter can include a bidirectional, steerable catheter tip configured to minimize injury to regions of the eye, including the suprachoroidal space (SCS). The ophthalmic catheter can include an asymmetric, double beveled tip geometry, and can be further configured to provide access to regions of the eye for surgical and/or therapeutic interventions.

An ophthalmic catheter navigational system can be constructed in accordance with the principles herein. The system can provide navigable access to local regions of an eye for surgical and/or therapeutic procedures. The system can include a catheter having a multidirectional, steerable tip. An electromechanical mount can be operatively connected to the catheter, directly or indirectly, for controlling selective deflection of the tip in a first or a second direction as the tip is advanced toward a target area, such as one or more local regions of the eye. The navigational system can be configured to form a delivery channel to access the target area, if desired. The delivery channel can be configured for access to and/or targeted delivery of therapeutic payloads to specific target areas, such as the SCS or other region/regions of the eye.

The system can enable delivery of larger therapeutic payloads than deliverable via conventional modalities to all regions of the posterior eye, and can provide access for a light source and/or laser and/or other surgical device while minimizing stress to the adjacent ocular tissues. The catheter of the system can further include a first side and a second side, wherein the steerable tip moves in a first direction toward the first side or a second direction toward the second side based on drive inputs. The drive inputs can be manual, mechanical, or deliverable via the electromechanical mount to at least one of the first side and the second side of the catheter. The system can further include a bendable sheath configured to selectively seat the catheter therein. Further, a catheter system in accordance with the principles herein can include a catheter configured to controllably navigate through regions of the eye, such as the SCS, or other regions of the human body. The catheter system can further include a sheath selectively connectable thereto. The sheath can be configured for access or delivery of a targeted therapeutic payload to regions of the eye, such as the SCS, upon removal of the catheter therefrom, if desired. The system can be configured to deliver a targeted therapeutic payload including one or more therapies for the treatment of one or more posterior eye diseases. The system can further include an electromechanical mount connected, directly or indirectly, to the catheter. The electromechanical mount can be configured to controllably navigate the catheter via controllable deflection of a catheter tip, wherein drive inputs operably connected thereto control the direction of the deflection of the catheter toward the regions of the eye, including posterior regions of the eye and/or the SCS.

Moreover, the systems can include a catheter having a width greater than a height to access/or deliver payload toward posterior regions of the eye, such as the SSC, wherein the larger width to height ratio allows for efficient displacement of tissue, thereby increasing the delivery channel payload while minimizing tissue displacement, and wherein the systems are comprised of disposable components. In certain embodiments, the tip can be further defined by a 2:4 aspect ratio or other various ratios. A system may be configured to provide access to and/or deliver therapies to posterior regions of the eye, such as the SCS configured in accordance with the principles herein can include a motor-driven bendable catheter having a flexible tip. A guidance/navigation system can be provided, and operably connected to the system for steering the flexible tip of the system to access an anatomical region, such as the posterior regions of the eye, adjacent tissue or orbit of the eye, or other suitable region wherein the system is configured to minimize ocular or other tissue damage. A steerable catheter tip and navigation system can be configured in accordance with the principles herein to controllably reach a target location and provide drug delivery and or surgical therapies to the target location that were not previously possible given the known systems. In some embodiments, a steerable catheter system can include a catheter including an asymmetric catheter tip for blunt dissection of tissues; a motor-driven control operably connected to the catheter to drive a catheter tip through tissues; and the system can be configured to selectively form a pathway to a target location. In certain embodiments, the catheter tip can be further defined by a beveled tip. In some other embodiments, the catheter tip can be further defined by a tip comprising one or more beveled surfaces. Thus, an asymmetric double bevel catheter tip system can be provided for greater catheter tip contact area preferentially with one tissue type, thus permitting a minimizing of pressure preferentially during a surgical procedure. The system can further include a hand-driven apparatus for steering the catheter tip. In some embodiments the system can further include a mechanical drive system for steering the catheter tip. In still other embodiments the system can further include a motor-driven drive system for steering the catheter tip.

In still other examples, systems constructed in accordance with the principles herein can comprise a bendable catheter. The catheter can be configured to bend via a bifurcated portion of a catheter shaft to produce two opposing members connected to a tip of the catheter. The opposing members, or sliding layers, can be shifted relative to one another, producing uniform, multidirectional flex of the tip via movement of the bifurcated portion of the catheter shaft. In addition, a steerable catheter tip system for an ophthalmic catheter can include a semi-rigid disposable sheath configured to selectively insert a portion of a catheter body therein. The sheath can be configured to constrain catheter flex of the portion of the catheter body inserted therein.

Other systems constructed in accordance with the principles herein can include a navigable catheter system. The catheter system can include an electromechanical mount housing, a servomotor, and a processor configured to receive inputs from a drive system. The mount can be configured to translate sliding layers of a catheter relative to each other. The mount can be operably connectable to the catheter and to the drive system. The system can include a sheath surrounding and constraining the catheter, wherein the sheath and the drive system, via the electromechanical mount, are configurable to control directional movement of a catheter tip of the catheter to steer the catheter tip. The sheath of the system can be configured to enable access to a target location. The system can be configured such that the therapeutic payloads are delivered to the target location through the sheath directly, or by guiding a syringe or other delivery device through the sheath. In certain embodiments, the catheter can be formed of a flexible, disposable material. In some embodiments, the drive system can further comprise at least one foot pedal, a manual drive guide, or a mechanical drive guide. The system can include a catheter including a shaft having a bifurcated portion, wherein relative sliding of the bifurcated portion of the catheter via the sheath during operation directs the movement of the catheter tip. The electromechanical mount system can be configured for precise and accurate control of a steerable catheter tip removably connected, either directly or indirectly thereto, in accordance with the principles herein. The mount system can comprise a housing configured to removably secure a sheath thereto, wherein the sheath is configured to guide bidirectional navigation of the catheter tip.

The system can be constructed to provide steering of the steerable catheter tip around any physiologic curvature and obstacles while reducing damage to tissue, and while providing access to a target region or location via the sheath or tissue separation channel. The system can further include a catheter body integrally formed with the catheter tip. The sheath can be further defined by a flexible sheath, removably connectable to the drive system via the mount.

In one embodiment according to the present disclosure, a catheter comprises a bifurcated shaft body, and a tip coupled to the bifurcated shaft body. The tip is configured to dissect a portion of a tissue in a controlled navigation for targeted delivery of a payload to an area of or within the tissue. In the addition, the tip is adapted to bend along a shape of the area of the tissue.

In another embodiment of the present disclosure, a catheter system comprises a catheter having a bifurcated shaft body and a tip coupled to the bifurcated shaft body. A sheath surrounds a portion of the bifurcated shaft body, and the tip extends outwardly from an end of the sheath. In addition, a navigation system is operatively coupled to the bifurcated shaft body of the catheter for controlling selective deflection of the tip of the catheter. So configured, the sheath is adapted to form a delivery channel to an area of tissue, and the delivery channel is configured for access and/or targeted delivery of a therapeutic payload to the area of tissue.

In yet another example, a method of operating a catheter system comprises providing a catheter with a bifurcated shaft body and a tip coupled to the bifurcated shaft body, and a sheath surrounding a portion of the bifurcated shaft body. The method also includes controlling selective deflection of the tip of the catheter by a navigation system operatively coupled to the bifurcated shaft body of the catheter to form a delivery channel configured for access and/or a targeted delivery of a therapeutic payload.

In still another example, a catheter tip system comprises an asymmetric double bevel tip adapted to be coupled to a shaft of a catheter. The tip is adapted to provide greater tip contact area preferentially with one tissue, permitting minimizing of pressure preferentially during a surgical procedure.

In some examples, the system may further comprise one or more of a hand-driven apparatus, a mechanical drive system, or a motor-driven drive system. In addition, each of the hand-driven apparatus, the mechanical drive system, and the motor-driven drive system are for steering the tip.

In another example, the system may comprise a bifurcated shaft coupled to the tip, and a sheath for receiving a portion of the bifurcated shaft, such that the tip outwardly extends from an end of the sheath and the bifurcated shaft body is disposed within the sheath, one or more of reducing flex of the bifurcated shaft body, isolating movement of the tip, and providing access and/or targeted delivery of a therapeutic payload to an area of tissue via the sheath.

In another form, one or more of the bifurcated shaft body and the sheath may comprise at least one biocompatible polymer having one or more of: (1) a hardness or an elasticity equal to a minimum value to minimize deflection; or (2) a geometry including a layer with an increased thickness.

In yet another form, the method may further comprise passing force through opposing longitudinal motion of two opposing members of the bifurcated shaft body via the navigation system, resulting in pivoting of the tip due to relative translated force differential between the two opposing members when the sheath is in place.

In another form, providing a catheter with a bifurcated shaft body and a tip coupled to the bifurcated shaft body, and a sheath surrounding a portion of the bifurcated shaft body may comprise providing the bifurcated shaft body and the sheath comprising biocompatible polymers having: (1) one or more of a hardness or an elasticity equal to a minimum value to minimize deflection; or (2) a geometry including a layer with an increased thickness.

### BRIEF DESCRIPTION OF THE DRAWINGS

These and other features and advantages of the various exemplary embodiments disclosed herein will be better understood with respect to the following description and drawings, in which:

FIG. 1A is perspective view of a conventional catheter tip having a blunt tip design.

FIG. 1B is a side view of the conventional catheter tip of FIG. 1A.

FIG. 1C is a perspective view of another conventional catheter tip having a olive tip design.

FIG. 1D is a side view of the conventional catheter tip of FIG. 1C.

FIG. 1E is a perspective view of a catheter tip according to one example of the present disclosure.

FIG. 1F is a side view of the catheter tip of FIG. 1E.

FIG. 1G is a perspective view of a catheter tip according to another example of the present disclosure.

FIG. 1H is a side view of the catheter tip of FIG. 1G.

FIG. 1I is a perspective view of a catheter tip according to another example of the present disclosure.

FIG. 1J is a side view of the catheter tip of FIG. 1I.

FIG. 2 is a perspective view of a catheter according to another aspect of the present disclosure, the catheter having a bifurcated shaft and movable from a resting position to a moved position, depicted by shading, in which force is applied to the bifurcated shaft.

FIG. 3 is a perspective view of a simulation setup for assessment of various catheter tip designs, with catheter tip depicted just before insertion into a portion of a tissue.

FIG. 4A is a perspective view of a catheter system according to another aspect of the present disclosure.

FIG. 4B is a perspective view of a portion of the catheter system of FIG. 4A.

FIG. 4C is a perspective view of a translational element of an electromechanical mount of the catheter system of FIG. 4A.

FIG. 4D is another perspective view of the translational element of the electromechanical mount of the catheter system of FIG. 4A.

FIG. 4E is a perspective view of a portion of the catheter system of FIG. 4B.

FIG. 4F is a perspective view of an enclosure to which the catheter is attached.

FIG. 4G is a perspective view of a collar adapted to be attached to a portion of the housing of FIG. 4D.

FIGS. 5A-5C are perspective view of articulation of a catheter of the present disclosure in an exemplary in vitro model.

FIGS. 6A-6C are perspective views of additional articulation of the catheter of FIG. 5, with the catheter ultimately withdrawn to leave behind a sheath in FIG. 6C.

FIGS. 7A-7H are perspective views of examples of catheter tip geometries according to the present disclosure.

FIG. 8 depicts a delivery channel formed for injection of a payload into a sprachoroidal space (SCS) of a human eye.

FIG. 9 is a block diagram depicting another catheter system in accordance with the present disclosure.

FIG. 10 is a perspective view of a manual controller that may be used with the catheter systems and catheters of the present disclosure.

FIG. 11 is another perspective view of the manual controller with a catheter of the present disclosure attached hereto.

FIGS. 12A and 12B are perspective views of a training model for the catheter system of the present disclosure.

### DETAILED DESCRIPTION

Although the foregoing text sets forth a detailed description of numerous different embodiments, it should be understood that the legal scope of the invention may be defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

Generally, a navigational catheter system has been developed for access and or the targeted delivery of payloads, including therapeutic treatments, to the any anatomical region, including the suprachoroidal space (SCS). The system consists of a customized catheter tip for blunt dissection of tissues, an electromechanical mount for controlled catheter navigation, and a method for targeted delivery of payloads. The catheter tip design was evaluated in dynamics simulations and its ability to deliver large payloads while minimizing stress to the adjacent tissues was quantified. A novel in vitro model of the eye was also designed and fabricated to demonstrate the capability of the catheter system to controllably navigate within the suprachoroidal space and deliver a targeted payload. This system enables the delivery of large therapeutic payloads to the SCS for the treatment of posterior eye diseases, thereby impacting the development and availability of vision-saving treatments.

The difficulty associated with navigating instruments through the SCS precludes clinical adoption and the availability of novel therapies for individuals with vision-threatening disease. Therefore, in accordance with the principles herein a catheter system including a catheter for use in the SCS is set forth. The catheter tip can be designed to minimize stress to the tissues defining the SCS and, in combination with incorporating features for controlled steerability, has the potential to deliver large payloads consistent with advanced therapies to targeted locations.

Specifically, a catheter system containing at least the following three components is set forth herein: 1) the catheter tip for blunt dissection of tissues, 2) a configuration for controlled catheter navigation, and 3) a configuration adapted and constructed for targeted delivery of large payloads. In one example, the catheter comprises a bifurcated shaft body and a bidirectional tip coupled to the bifurcated shaft body. The bifurcated tip is configured to dissect a portion of a tissue in a controlled navigation for target delivery of a payload to an area of the tissue. In addition, the bidirectional tip is adapted to bend along a curvature of the area of the tissue.

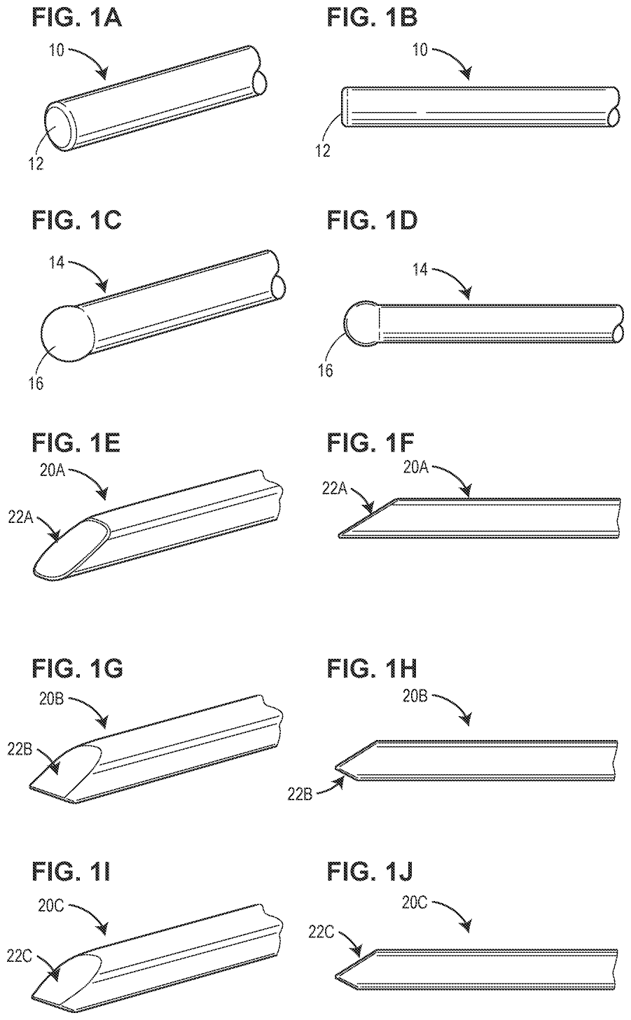

More specifically, and referring now to FIGS. 1E-1J, a number of different catheter tips can be constructed in accordance with the principles herein. As depicted in FIGS. 1E and 1F, and in one example, a portion of a catheter 20A includes a beveled tip 22A with a single bevel. As depicted in FIGS. 1G and 1H, a portion of a catheter 20B includes a tip 22B having a asymmetric double-bevel. In yet another example depicted in FIGS. 1I and 1J, a catheter 20C includes a tip 22C also having an asymmetric double-bevel and an additional taper compared to tip 22B of FIGS. 1G and 1H. These three exemplary tip designs for use in the SCS are described in detail herein, although other designs can be achieved in accordance with the principles herein. In addition, while all designs may be used in the SCS area, for example, it will be understood that all designs may alternatively be used in various other tissues and/or areas of a human body and still fall within the scope of the present disclosure, as explained more below.

Further, all three exemplary designs were derived from a common 1:1 cross-sectional ratio (height:width). To facilitate blunt dissection at the catheter tip, all tips 22A, 22B, and 22C featured the beveled design. As further explained below, each tip 22A, 22B, and 22C is adapted to bend along a shape of an area of a tissue, such as a curved or a non-curved portion of the tissue, including the SCS. More generally, each tip 22A, 22B, 22C is configured to provide access to regions of a human body for surgical and/or therapeutic interventions. In addition, the asymmetric double bevel of tips 22B and 22C of catheters 20B and 20C, respectively, were designed to provide greater tip contact area with the choroid as opposed to the sclera. This difference in contact is meant to permit a greater distribution of SCS separation forces along the choroid, to decrease effective pressure and minimize potential damage to the vasculature.

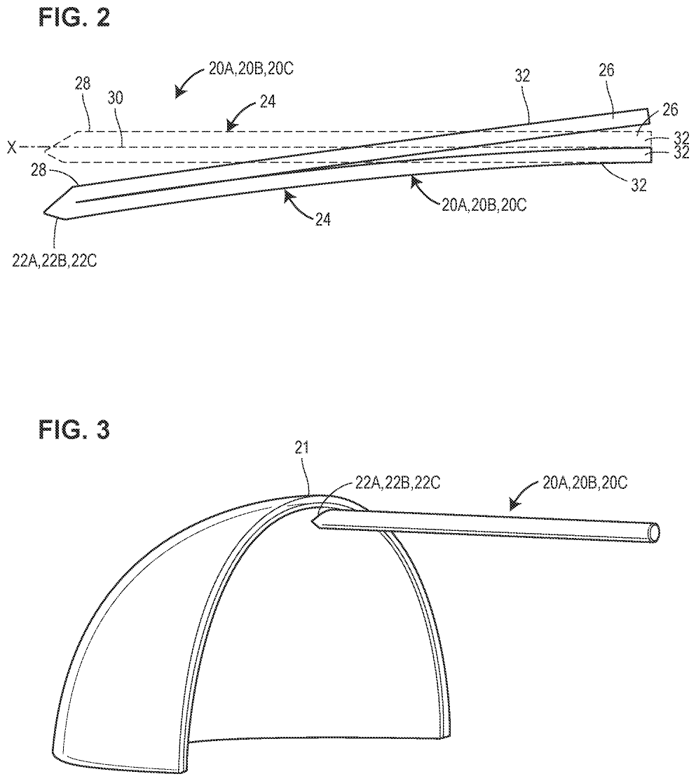

Referring now to FIG. 2, the catheter 20A, 20B, and 20C of FIGS. 1C-1E, respectively, is depicted. Each catheter 20A, 20B and 20C includes a bifurcated shaft body 24 to which the tip 22A, 22B, and 22C is coupled. In addition, the bifurcated shaft body 24 includes a proximal end 26, a distal end 28, and a slit 30 extending along a length of the bifurcated shaft body 24. Specifically, the slit 30 extends from a position near the distal end 28 and through the proximal end 26, such that the proximal end 26 includes a pair of opposing members 32 that are coupled to the tip 22A, 22B, 22C at the distal end 28. Said another way, the bifurcated shaft body 24 may include a pair of opposing layers 32, and the opposing layers 32 form sliding members 32 adapted to shift relative to each other. As a result, a uniform flex along the length of the shaft body 24 is produced. In one example, the sliding members 32 may shift one or more of axially or longitudinally relative to each other and/or a longitudinal axis X of the bifurcated shaft body 24.

Catheter navigation through the SCS can be achieved in accordance with the principles herein as being accomplished by a system configured to accommodate the combined actions of catheter tip articulation and driving of the whole catheter. Catheter articulation can be accomplished, for example, via longitudinal bifurcation of the bifurcated shaft body 24 of the catheter 20A, 20B, 20C to produce the two opposing members 32 connected to the catheter tip 22A, 22B, 22C. These opposing members 32, which may also be referred to as “sliding layers”, can be shifted relative to each other, producing uniform flex of the entire catheter. While FIG. 2 depicts the standard blunt-tipped catheter, any of the beveled catheter tips 22A, 22B, and 22C may be used within the context of the present disclosure.

Catheter articulation can also be achieved, for example, via a system configured to accommodate the catheter tip 22A, 22B, 22C articulation through a uniform flex of the entire bifurcated shaft body 24 of the catheter 20A, 20B, 20C. In this exemplary embodiment, a catheter tip 22A, 22B, 22C can articulate separate from the bifurcated shaft body 24 via the sheath 34 while the bifurcated shaft body 24 can flex uniformly at the same time.

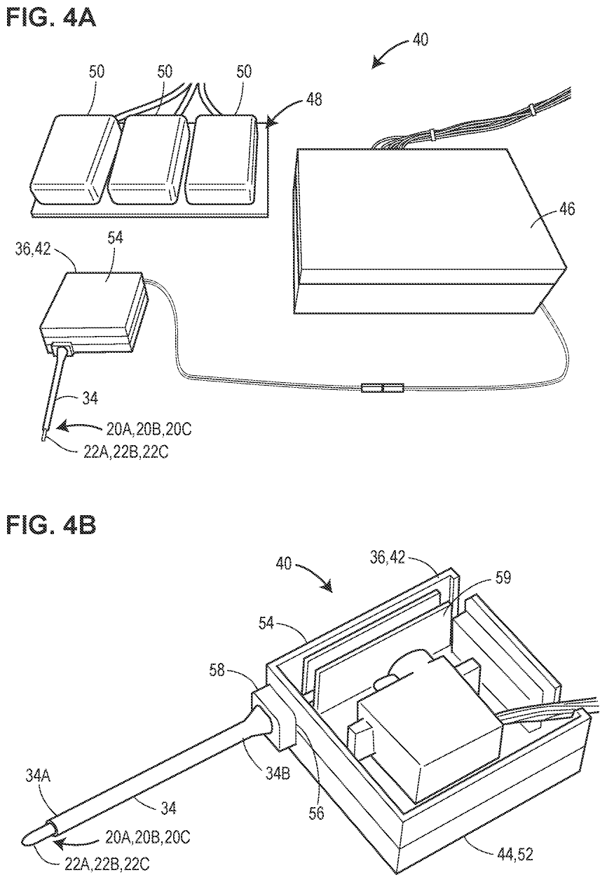

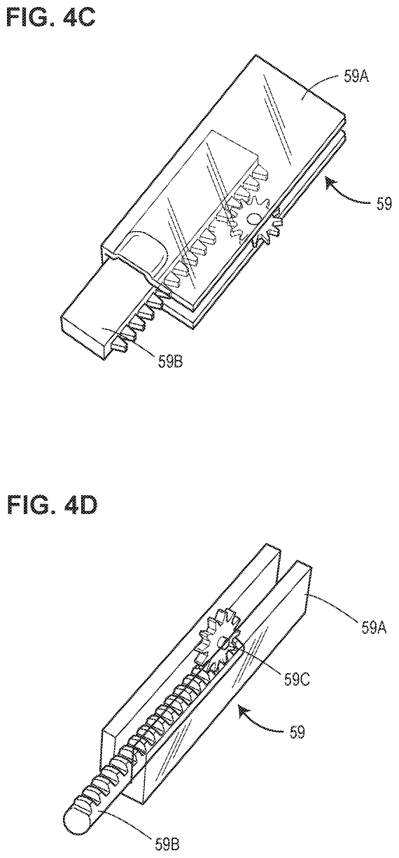

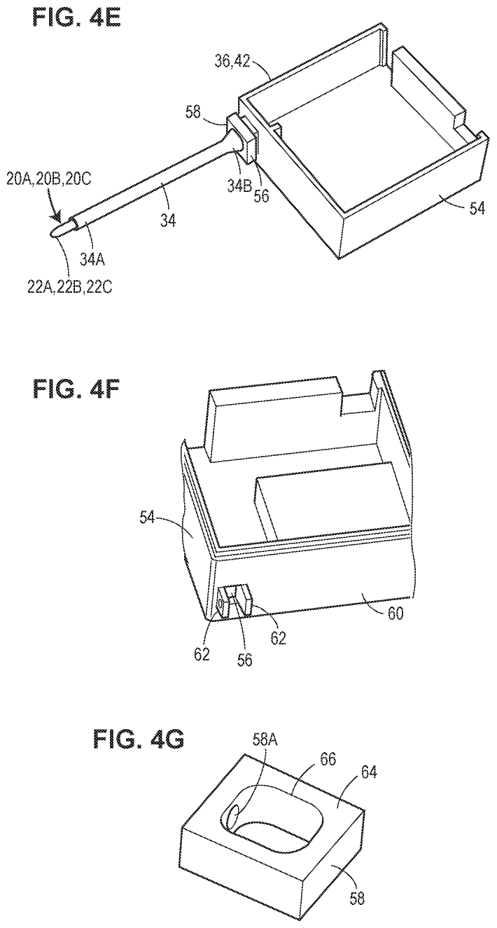

In one exemplary embodiment, and as depicted in FIGS. 4A and 4B, for example, a semi-rigid sheath 34 can be provided about the shaft body 24 to span the length of the catheter 20A, 20B, 20C, which greatly reduces catheter flex in favor of isolated tip articulation. Notably, in this design, the sheath 34 would also enter the SCS, and therefore must be flexible enough to bend with variable eye curvature or a curved or non-curved portion of another area within the human body. Specifically, when a portion of the catheter 20A, 20B, 20C is disposed within the sheath 34, the tip 22A, 22B, 22C is adapted to move separate from the bifurcated shaft body 24 via the sheath 34 and in a direction about the longitudinal axis X (see, e.g., FIG. 2) of the bifurcated shaft body 24. At the same time, the bifurcated shaft body 24 uniformly flexes relative to the longitudinal axis X of the bifurcated shaft body. As further depicted in FIGS. 4A and 4B, the tip 22A, 22B, 22C outwardly extends from a distal end 34A of the sheath 34 when the shaft body 34 is disposed in the sheath 34. Thus, the sheath 34 helps reduce flex of the bifurcated shaft body 34 and isolate movement of the tip 22A, 22B, 22C. The sheath 34 also provides access and/or targeted delivery of a therapeutic payload to the area of the tissue, as explained more below.

As is also understood from at the least the foregoing and explained more below, the catheter 20A, 20B, 20C is used for accessing regions of an eye or other area of the human body for surgical and/or therapeutic interventions. In addition, the tip 22A, 22B, 22C is adapted to minimize injury to regions of the eye, including the SCS, or other areas of the human body.

Referring still to FIGS. 4A and 4B, additionally, to facilitate precise and accurate control of tip articulation, a navigation system 36 is provided. The navigation system 36 is operatively coupled to the bifurcated shaft body 24 of the catheter 20A, 20B, 20C for controlling selective deflection of the tip 22A, 22B, 22C of the catheter 20A, 20B, 20C. Thus, together the navigation system 36, the catheter 20A, 20B, 20C, and the sheath 34 surrounding a portion of the bifurcated shaft body 24 form a catheter system 40 of the present disclosure. As explained above and more below, the sheath 34 forms a delivery channel to an area of tissue in the human body, such as the SCS, and the delivery channel is configured to access and/or targeted delivery for a therapeutic payload to the area of tissue.

In one example, and as depicted in FIGS. 4A and 4B, the navigation system 36 includes an electromechanical mount 42. The electromechanical mount 42 translates the sliding layers 32 of the bifurcated shaft body 24 (FIG. 2) of the catheter 20A, 20B, 20C relative to each other using a suitable driving mechanism 44, such as a motor, a servomotor or the like. Through combined use of the sheath 34 and electromechanical mount 42, catheter flex would be refined to precise, accurate, and controllable articulation of the tip 22A, 22B, 22C of the catheter 20A, 20B, 20C. More specifically, motion of the tip 22A, 22B, 22C is isolated via the sheath 34, and the eletromechanical mount 42 controls articulation of the tip 22A, 22B, 22C.

As depicted in FIG. 4A, the electromechanical mount 42 is also operatively coupled to a control box 46 and a drive system 48. In one example, the drive system 48 includes a plurality of foot pedals 50. In other examples, the drive system 48 may include one or more buttons or another actuating member and still fall within the scope of the present disclosure.

As depicted in FIG. 4B, the electromechanical mount 42 houses the motor 44, such as a servomotor, and a processor 52 that is configured to receive inputs from the drive system 48. The electromechanical mount 42 also includes an enclosure 54 having an opening 56 with a collar 58, and the sheath 34 is coupled to the collar 58 along with a translational element 59.

Referring now to FIGS. 4C and 4D, the translational element 59 of the eletromechanical mount 42 is depicted. The translational element 59 consists of three parts: one static housing 59a, one linear gear 59b, and a rotational gear 59c. The linear and rotational gear are in a rack and pinion orientation. In one example, one layer 32 of the bifurcated catheter 20A, 20B, 20C is slid into the static housing 59a and the other into the linear gear 59b. As the linear gear 59b is driven by the rotational gear 59c (e.g., via the servo motor), the catheter layers 32 slide relative to themselves and generate flex/tip articulation.

More specifically, and referring now to FIGS. 4E-4G, the opening 56 of the enclosure 54 of the electromechanical mount 42 is disposed on one end of a side wall 60 of the enclosure 54. A pair of tabs 62 outwardly extend from the opening 56, as depicted in FIG. 4F, and are adapted to engage the collar 58. As depicted in FIG. 4G, and in one example, the collar 58 includes a rectangular housing 64 with a central aperture 66 that receives a proximal end 34B (FIGS. 4B and 4E) of the sheath 34 for securement to the enclosure 54. While the collar 58 is an exemplary collar 58 to hold the sheath 34 in place, these components may also be used to attach a syringe (not shown) to flex tubing for delivery of therapeutics in other examples and still fall within the scope of the present disclosure.

In one example, the collar 58 can anchor the sheath 34 to the case 54 using a series of mating dimples 58a (FIG. 4G) or other suitable securing components or structure. Initially, the sheath 34 can be slit longitudinally, such as approximately 4-5 mm for certain applications (some posterior eye procedures, for example), and the resulting two members 32 can be slid over two opposing projections from the case, or other locking structure. The collar 58 can then be slid over the sheath 34 until it mates with the case or the enclosure 54 (e.g., via the dimples 58a), effectively pinching the sheath 34 in place. For example, FIG. 4G depicts the collar 58 with one female dimple 58a in view circled (only one shown in view). FIG. 4F depicts the case 54 with opposing projections or tabs 62 and male dimples. In one example, a slight slit can be provided in the sheath 34 to allow the sheath 34 to straddle the opposing dimpled projections or tabs 62 from the case or enclosure 54, for example. A tubing could also be attached to a modified syringe (not shown) using the same or similar collar, and could serve as a basis for an accessory of the device.

As noted above, and in one exemplary embodiment, the catheter 20A, 20B, 20C and sheath 34 can be configured for the delivery of therapeutics or other payloads. For example, after insertion of the sheathed catheter into the SCS, it can be navigated to a desired location. The catheter 20A, 20B, 20C can then be removably withdrawn from the sheath 34, leaving the sheath 34 in place to serve as a direct port to the desired location within the SCS, as explained more below. The operator can then deliver a payload through the sheath 34 by either using the sheath 34 directly, or by guiding a primed syringe (not depicted) or other drug delivery device through the sheath 34.

Referring to FIG. 3, the catheter models, such as catheter computer models, similar to catheters 20A, 20B, and 20C, were scaled appropriately for the eye and placed at a consistent position just prior to insertion into an area of tissues 21, such as the SCS. The eye was assumed to be fixed at the flat planar surfaces along the rim of the scleral outer layer. A pressure of 2 kPa (equivalent to 15 mmHg) was applied to the inner chorioretinal surface, comparable to normal intraocular pressure in the human eye. An initial force was used to pull a section of the chorioretinal tissue near the catheter 20A, 20B, 20C away from the sclera so that a catheter head could be advanced into the space unimpeded. This force was immediately removed after catheter insertion. To drive the catheter, a remote displacement condition at 5 cm/s was applied to the planar surface at the rear of the body. All other translational and rotational motions at the rear surface were fixed. This effectively directed the catheter through the SCS as a surgeon would, holding a section of the catheter body rigid and driving it forward.

Nodal measurements of equivalent stress, equivalent strain, and deformation were calculated and recorded for all three bodies and at each time step. Data used for comparative analysis of catheter tip designs were taken from the last time point, after full insertion of the catheter into the SCS. Data was only evaluated from nodes where model deformation was >1 mm, corresponding to regions of the model that were impacted by catheter insertion.

A novel in vitro model was created to assess the ability of the navigational catheter system to reach and deliver a payload to a targeted location. This model represents the whole eye at customizable scale. This model mimicked the SCS as an interface between the membrane (chorioretinal complex) and ballistics gel (sclera). Access to the SCS analog was available following incision through the scleral analog, and without puncture to the chorioretinal analog. Internal pressure within the model was controlled using a cannula attached to a water-filled reservoir on an adjustable-height post. The model was also produced in a variant that included a spatial restriction on the fluid-filled membrane. This restriction provided non-uniform curvature to the model, analogous to curvature changes that can occur in human eyes, and represented a navigational challenge for evaluation of the catheter system.

When delivering payloads to the posterior eye through the SCS, the ideal solution would maximize payload delivery potential while minimizing induced stress.

Therefore, a catheter stress-payload ratio (SPR) was defined, in units of Pascal/mm², as:

SPR=α/(A),

where α is the mean stress induced on the SCS along the catheter length, and A is the cross-sectional area of the catheter shaft. Thus, in general, lower values of SPR indicate a decreased induced stress on opposing tissues and/or an increased cross-sectional area corresponding to a greater payload delivery potential. ANSYS mesh convergence was determined by unpaired t-test of SCS stress from repeated simulations using the blunt-tipped catheter with small and large mesh sizing for all bodies. The mean and standard deviation of SCS stress data were calculated for each catheter tip design. The SPR was calculated using mean SCS data and known catheter cross-sectional areas from the Solidworks computer models. ANOVA with Tukey post hoc was used to determine statistical significance between catheter tip designs. Statistical analysis was performed using SPSS (IBM Corporation, New York, USA; Version 26) and statistical significance was accepted at P<0.05.

There was no statistically significant difference in SCS stress data from repeated blunt-tipped catheter simulations with varied mesh sizes (P=0.88), indicating mesh convergence in the ANSYS simulation computer model.

A number of materials can be used to form suitable forms of one or more of the catheters, 20A, 20B, 20C, the catheter tips 22A, 22B, 22C, and/or the sheath 34 components in accordance with the principles herein. The materials require a certain amount of flexibility, prescribed for a particular use. The materials may have certain limitations and additional requirements for human or animal procedures.

Results from various tests of catheters, e.g., using computer models of the catheters, with various designs described above are provided in Tables 1-4 below.

TABLE 1

| | Mean | Standard | Cross-Sectional | Stress-Payload |
|---|---|---|---|---|
| Catheter | Stress | Deviation Stress | Area | Ratio |
| Tip | (Fa) | (Pa) | (mm²) | (Pa/mm²) |
| A | 31429 | 13104 | 5.28 | 5952 |
| B | 26283 | 10901 | 5.28 | 4978 |
| C | 23500 | 10591 | 5.28 | 4451 |
| Blunt | 32467 | 11256 | 4.91 | 6612 |
| Olive | 25356 | 7812 | 4.91 | 5164 |

TABLE 2

| | | Catheter Tip | | | | |
|---|---|---|---|---|---|---|
| Metric | Reference | A | B | C | Blunt | Olive |
| SCS | A | — | −5146** | −7929** | NS | −6073** |
| Stress | B | — | — | −2783* | 6184** | NS |
| (Pa) | C | — | — | — | 8967** | NS |
| | Blunt | — | — | — | — | −7111** |
| | Olive | — | — | — | — | — |

TABLE 3

| Catheter | Mean | Standard | Cross-Sectional | Stress-Payload |
|---|---|---|---|---|
| Tip and | Stress | Deviation Stress | Area | Ratio |
| Ratio | (Pa) | (Pa) | (mm²) | (Pa/mm²) |
| C (1:1) | 23500 | 10591 | 5.28 | 4451 |
| C (1:1.5) | 26722 | 11783 | 8.4 | 3181 |
| C (1:2) | 25010 | 11109 | 11.53 | 2169 |

TABLE 4

| | | Catheter Tip and Ratio | | |
|---|---|---|---|---|
| Metric | Reference | C (1:1) | C (1:1.5) | C (1:2) |
| SCS Stress (Pa) | C (1:1) | — | 3222** | NS |
| | C (1:1.5) | — | — | NS |
| | C (1:2) | — | — | — |

Specifically, table 1 provides simulated SCS stress data from catheter tip designs, catheter cross-sectional areas, and resulting SPR, with catheter tips A, B, and C in the table corresponding to the tips 22A, 22B, and 22C of the catheters 20A, 20B, and 20C provided in the foregoing description in each of Tables 1-4. There was a statistically significant difference in SCS stress between catheters (P<0.001). Differences in mean SCS stresses between catheter tip design are provided in Table 2. Briefly, tips C and olive-shaped tips provided less stress to the SCS than the tip ‘A’ and the blunt tip (P<0.001). While there was no statistically significant difference in SCS stress between the tips B, C, and the olive-shaped tip, the difference between the tips C and B approached statistical significance (P=0.062). The SPR revealed differences between the tips B, C, and olive-shaped tip, with the tip C being 10.9% and 13.8% lower than the tip B and the olive-shaped tip, respectively.

The effect of catheter cross-sectional ratio on SPR was assessed. Since the catheter tip C had the lowest SPR, simulations of that tip C design at different ratios were repeated, as shown in Table 3. There was a statistically significant difference in SCS stress between ratios (P=0.05). Differences in means between SCS stresses from simulations of catheter tip C at multiple cross-sectional ratios are provided in Table 4. Briefly, SCS stress was statistically significantly greater with the 1:1.5 ratio than the 1:1 ratio (P=0.04), although there was no statistically significant difference in SCS stress between 1:1 and 1:2 ratios. However, the SPR revealed considerable differences between ratios, with 1:2 being 51.2% and 31.8% lower than 1:1 and 1:1.5, respectively.

In one example, the catheter tip C in a 1:2 ratio was selected for fabrication due to its ability to impart minimal stress in simulations and its greatly reduced SPR. The catheters 20A, 20B, 20C can be fabricated from any suitable medical grade material or combination of materials. For example, in one example, any of the catheters 20A, 20B, 20C can be fabricated by casting a two-part urethane compound at either 85 A or 50 D Shore hardness (Smooth-On Inc, Pennsylvania, USA; Models: Simpact 85 A and Task 14, respectively) into a customized three-piece mold. The mold can consist of two mating blocks configured to produce catheters at 75 mm in length with a cross section of 2×4 mm (2× scale), for example. The bifurcation through the shaft body 34, such as the slit 30, can be 0.1 mm in thickness, and can extend through the catheter length, ending 4 mm from the catheter tip 22A, 22B, 22C (see, e.g., FIG. 2), if desired. Exemplary catheters can be fabricated to scale for human use, or larger or smaller for animal therapies as needed.

Further, and in yet another example, the catheter sheath 34 was fabricated by casting a two-part urethane compound at 50 D Shore hardness into a customized mold. The mold consisted of two concentric extruded ovals and was 3D printed from an FDM printer (Prusa Research, Prague, Czech Republic; Model: MK3S) using water-soluble polyvinyl alcohol filament. After curing, the mold was placed in a perturbed water bath to release the sheath. Sheaths were fabricated with a major axis of 4.5 mm, a minor axis of 2.5 mm, and were approximately 0.7 mm in thickness. In this example, the sheath 34 extended the length of the catheter shaft body 34, leaving 8-10 mm of the tip 22A, 22B, 22C exposed.

In addition, the electromechanical mount 42 may be 3D printed using a polylactic acid filament and consisted of a translational and fixed element, which attached to the opposing layers 32 of the catheter 20A, 20B, 20C. The translational element was connected to the servomotor 44 (Tower Pro Pte Ltd, China; Model: SG90) and programmed using an Arduino microprocessor (Arduino, Ivrea, Italy; Model: Arduino Uno) to provide relative motion of the opposing layers 32. In this example, the servomotor position was controlled by a series of foot pedals 50 (URBEST; Model: TFS-01) to incline or decline the catheter tip 22A, 22B, 22C in adjustable angle increments. The rotation from the servomotor 44, coupled to the electromechanical mount 42, resulted in a ±35 degrees range of catheter tip 22A, 22B, 22C articulation about the neutral position. With the adjustable angle increment set to 5 degrees of servomotor rotation per foot pedal actuation, the catheter tip 22A, 22B, 22C achieved a resolution of 2.5 degrees/step over a central range of ±30 degrees.

The customized enclosure 54 was designed and constructed to anchor the electromechanical mount 42, the servomotor 44, the catheter 20A, 20B, 20C, and the sheath 34 into a single, space-efficient profile forming the catheter system 40. In one example, the enclosure 54 is a case printed using a stereolithography 3D printer (Formlabs Inc, Massachusetts, USA; Model: Form 2 with Clear RS-F2-GPCL-04 resin). The sheath 34 was anchored to the case 54 using the customized press-fit collar 58, which, when removed, allowed decoupling of the sheath 34 from the case 54 for the delivery of therapeutics.

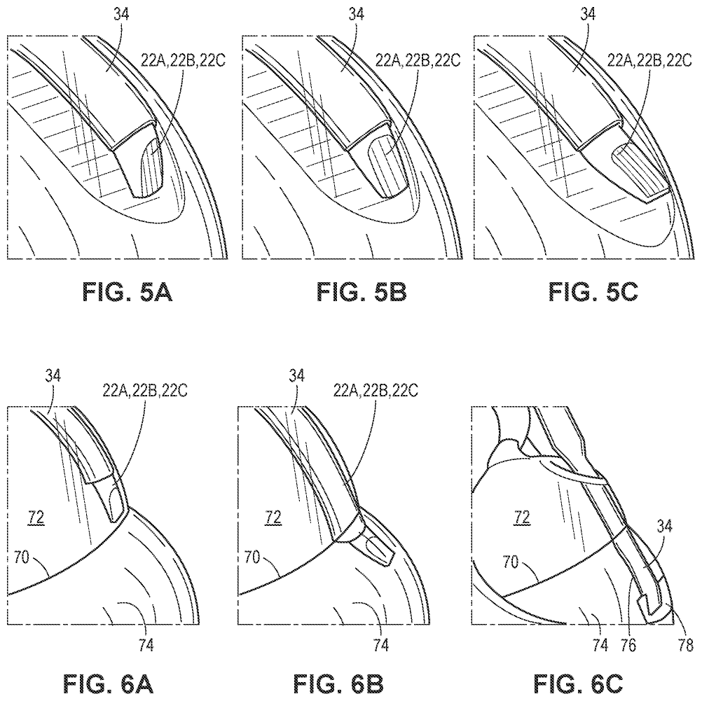

Referring now to FIGS. 5A-5B, an in vitro eye model was fabricated at 2× scale (60 mm diameter), consistent with the scale of the catheter 20A, 20B, 20C. The model was then cannulated and internal pressure was set between 10 and 21 mmHg, comparable to normal intraocular pressure in the human eye. Insertion of the sheathed catheter 20A, 20B, 20C into the membrane-gel interface was performed through a 3-5 mm incision on the external face of the model, roughly 15 mm superior to the equator. The sheathed catheter 20A, 20B, 20C was then driven through the interface to generate an SCS analog through blunt dissection of the membrane from the ballistics gel. Combined action of catheter tip articulation and drive was used to successfully navigate along the curvature of the model through the interface. FIGS. 5A-5C show the catheter 20A, 20B, 20C and the sheath 34 of the catheter system 40 embedded in the model and at varying catheter tip articulations. Specifically, FIG. 5A depicts the catheter tip 22A, 22B, 22C at a maximum inward articulation, internally articulated toward a chorioretinal analog, in this example. FIG. 5B depicts the catheter tip 22A, 22B, 22C articulating to match a curvature of the model, and FIG. 5C depicts the catheter tip 22A, 22B, 22C at a maximum outward articulation, externally articulated toward a scleral analog, in this example. Notably, failure to appropriately articulate the catheter tip 22A, 22B, 22C during navigation resulted in bunching against one material or the other and subsequent stalling.

Referring now to FIGS. 6A-6C, the catheter 20A, 20B, 20C was also navigated through the model with spatial restrictions imposed on the fluid-filled membrane. Specifically, the model includes a spatial restrictor 70 disposed between a fluid-filled membrane 72 and a ballistics gel 74. The local changes in curvature caused by the restrictor 70, which may include any obstacle, were impossible to overcome without catheter articulation, as repeated attempts resulted in stalling and the inability to drive the catheter 20A, 20B, 20C farther. After articulation and navigation beyond the restrictor 70, as depicted in FIG. 6B, the mechanism for delivery of therapeutics was successfully evaluated. The collar 58 was removed and the catheter 20A, 20B, 20C with case 54 were retracted while the sheath 34 was held in place. The remaining sheath 34 provided a direct port 76 to a targeted location 78 in the model, as depicted in FIG. 6C. A syringe was used to inject red dye through the sheath 34, which validated the potential of the catheter system 40 for targeted delivery of a therapeutic.

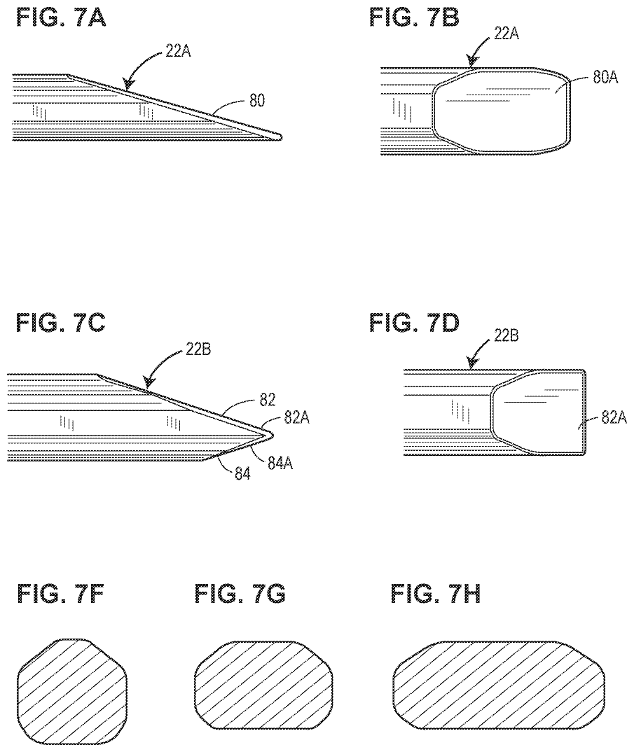

Referring now to FIGS. 7A-7H, several example geometries for various tips 22A, 22B, 22C of the catheter 20A, 20B, 20C are provided. Specifically, FIGS. 7A and 7B include a single bevel tip 22A, with a first side 80 and a first surface area 80a. In addition, FIGS. 7C and 7D include a double beveled tip 22B having a first side 82 with a first surface area 82a and a second side 84 with a second surface area 84a. The first surface area 82a is greater than the second surface area 84a, such that the first surface area 82a has a contact area greater than a contact area of the second surface area 84a. The first and second surface areas 82a, 84b are adapted to contact tissues during use. As a result, the first surface area 82a will contact more tissues during use than the second surface area 84a, for example. In addition, the first side 82 of the double beveled tip 22B includes a first length and the second side 84 includes a second length, and the first length is longer than the second length. As depicted in FIGS. 7F-7H, various other shapes and geometries may be used for a tip of the catheter 20A, 20B, and 20C and still fall within the scope of the present disclosure.

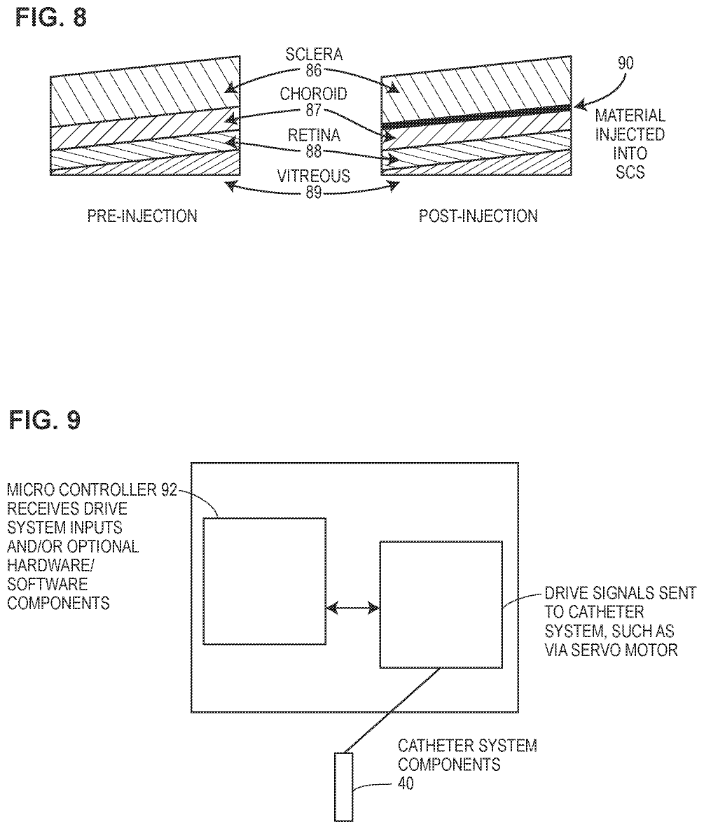

Referring now to FIG. 8, an exemplary portion of SCS is depicted both before an injection and after an injection. As noted therein, the SCS includes several parts, the sclera 86, the choroid 87, the retina 88, and the vitreous 89. The catheter system 40 is able to form a delivery channel 90, which includes material after an injection, as depicted in FIG. 8.

A preferential catheter tip design was determined using computer-based simulations. Five catheter tips and an eye model were designed and 3D modeled using computer-aided design software and exported to ANSYS for dynamics simulations, where the catheters were inserted into, and navigated through, the simulated SCS. Stress data from the choroidal and scleral tissues were exported to assess the effect of catheter tip design. Furthermore, the relative ability of a catheter to deliver greater payloads while minimizing stress to the neighboring opposing tissues was quantified using the stress-payload ratio (SPR). Catheter tip 22C and the olive-shaped catheter tip induced the least stress, but the tip 22C provided the lowest SPR by more than 10%. The tip 22C was then selected to determine the effect of catheter cross-sectional area on the SCS stress and SPR. There was no statistically significant difference in stress between ratios 1:1 and 1:2 and both were lower than 1:1.5, but the 1:2 ratio had a greatly reduced SPR. These results indicate that the tip 22C at a 1:2 ratio was the most effective tip design for maximizing payload capacity. In this example, catheter tip design 22C at a 1:2 ratio was selected for fabrication and later evaluation.

Catheter navigation was performed by catheter tip articulation and drive of the whole catheter 20A, 20B, 20C. Catheter articulation was accomplished through bifurcation of the shaft 24 and the relative sliding of the resulting opposing layers 32 (FIG. 2). Articulation was confined to the catheter tip 22A, 22B, 22C alone through the use of the sheath 34, which extended the length of the catheter 20A, 20B, 20C. The electromechanical mount 42 was designed and implemented for precise and accurate control of catheter tip articulation, and the customized enclosure case 54 was developed to protect and coordinate all components. Bifurcated catheters 20C with the tip 22C in a 1:2 ratio were assembled into the catheter system 40.

To demonstrate the ability for the navigational catheter system 40 to reach and deliver a payload to a targeted location, a customized in vitro model was developed to represent the eye and SCS. The sheathed catheter 20A, 20B, 20C was inserted into the model and navigated to a targeted location. Targeted delivery of a payload was demonstrated using the sheath 34 as a port 76. Moreover, navigation and payload delivery were evaluated in models with a spatial restrictor, such as the restrictor 70 of FIGS. 6A-6C, presenting abrupt and severe changes to curvature. Use of these models demonstrate the ability of the catheter system 40 to achieve isolated catheter tip articulation and indicate that controlled articulation over the curvature or other non-curved area is feasible. Furthermore, it was determined that tip articulation is essential for navigation around curvature and other obstacles, and that the delivery of a payload to a targeted location using the sheathed catheter 20A, 20B, 20C is possible.

Material properties of the choroid and sclera in dynamic simulations were based on studies using enucleated eyes and may differ from in vivo material properties of the SCS. Further, materials for the in vitro model were selected to prioritize visualization of the catheter 20A, 20B, 20C during navigation and only qualitatively represented the SCS. Additional studies using the catheter system 40 in ex vivo and animal models can establish more details regarding the ability of the system to navigate and deliver payloads into the SCS. Herein details are set forth for the design and preliminary evaluation of the catheter system 40 for the targeted delivery of payloads. This system 40 may enable the delivery of large therapeutic payloads to the SCS for the treatment of ocular disorders, thereby impacting the development and availability of vision-saving treatments.

More generally, a wide variety of catheter tip sizes can be provided and selected based on a particular subject and anatomical features thereof, the surgical procedure to be performed, subject characteristics, i.e., whether human or animal subject, and what materials or devices are required to perform a particular procedure.

Any suitable sheath or layers of sheaths can be formed in a variety of ways in accordance with the principles herein. For example, the sheath 34 can be formed of a porous or non-porous material. Either material can be customized to provide a targeted or distributed delivery device for any given indication. The drive mechanism can be detached, the catheter 20A, 20B, 20C removed from the system, and a syringe can then be attached to the sheath 34 and materials delivered, inserted, or deposited therethrough, if desired. The sheath 34 can be flexible and disposable.

The sheath 34 can further include a disposable or non-disposable liner to accommodate passage of, for example, therapeutics, evacuation, biopsy or cauterization devices, a balloon and the like for a particular surgical procedure. For example, the catheter system 40 can provide a tissue separating port for a number of surgical procedures, such as bladder cancer, neurosurgical, or cardiac surgical procedures to name a few. A shaft of the sheath 34 can be configured with a reinforcing geometry such as a braid, a coil, or a slit tube that mimics a coil and combinations of the foregoing. The sheath 34 and/or the catheter 20A, 20B, 20C can incorporate or accommodate a light source, if desired.

Alternates for an electromechanical motor discussed in conjunction with certain embodiments include a servo motor, a linear actuator, a stepper motor or other suitable driver, and or a mechanical, hydraulic or pneumatic drive mechanism.

System components can include noise/vibration reduction features and components, if desired. Further, the length of a catheter can be varied to accommodate the needs of a particular procedure and location.

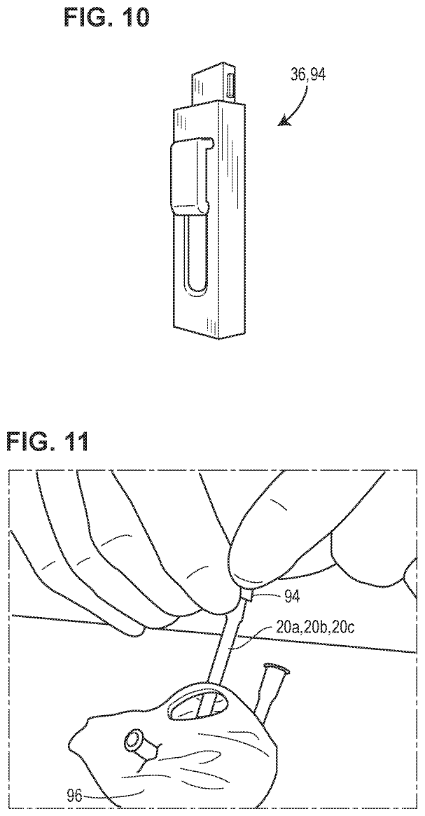

As depicted in FIG. 9, hardware and/or software components 90 can be provided in the catheter system 40 in order to assist or automate certain aspects of a surgical procedure. In addition, drive signals may be sent to the catheter system 40, such as via a servomotor. The system 40 could be configured to measure conductivity or impedance, if desired. Further, the actual curvature of the tip 22A, 22B, 22C as it bends could be monitored and displayed on a suitable display. In certain embodiments, the curvature of the tip 22A, 22B, 22C can be driven by a foot pedal 50, dial, a button or other actuating member and still fall within the scope of the present disclosure. In other embodiments, the system 40 can include components for mapping and driving the catheter 20A, 20B, 20C in a human hand-controlled system, or via a fully automated software system. The main catheter driving components of the device can be formed of medical grade disposable materials, if desired. Additionally, the system 40 can be configured to advance the shaft 24 of the catheter 20A, 20B, 20C in real-time based on system feedback, if desired.

The catheter system 40 may include a flexible catheter 20A, 20B, 20C and the sheath 34 removably disposed thereon. The sheath 34 can be selected to flex in response to the movement of a catheter driving mechanism, and can be selected to flex less than the flexible catheter 20A, 20B, 20C. The driving mechanism can be operated selectively via a dial or foot pedal or other suitable activation mechanism, if desired.

As explained above, the driving mechanism can be housed in a drive mechanism housing or enclosure 54. The sheath 34 can be selectively secured to the drive mechanism housing via a collar or other securing mechanism, if desired. The collar 58 can form a secure and selectively removably connection to the drive mechanism via any suitable connection configuration. For example, the collar 58 can include one or more anchoring protrusions formed to selectively mate to tabs 62 extending from the drive mechanism. In other embodiments, the collar 58 can incorporate a mated threading configuration, or other suitable configuration, for selectively securing the sheath 34 to the drive mechanism and/or a syringe or other device required for a particular surgical procedure. An adapter can be provided for connecting the sheath 34 to a luer lock connection. Further, the sheath 34 can be heparinized or suitably pre-treated to incorporate anti-clotting features and aid delivery of certain substances to the target area.

Software associated with the system could include, for example: providing feedback regarding position, visual sensation, electric signals or could include a position encoder for precision, or any other suitable function that assists the catheter systems 40 during a surgical procedure.

Custom models can be configured to mimic different anatomic variations or locations for the catheter, while the driver housing can be customized and configured to optimize operability and grip features, if desired. For example, a linear profile for the mount or driver could be easier to handle for certain surgical procedure, while ergonomic designs could also be beneficial in certain embodiments.

Referring now to FIGS. 10 and 11, another navigation system 36 may include a manual controller 94. The catheter 20A, 20B, 20C may be selectively inserted into the manual controller 94. The manual controller 94 may include both stationary and translating elements, and the translating elements are controlled by a user's fingers, as depicted in FIG. 11, for example. Specifically, in FIG. 11, the manual controller 94 has the catheter 20A, 20B, 20C attached thereto, which is being manually inserted by the surgeon into a pig eye 96. The user manually drives the direction and advancement of the catheter 20A, 20B, 20C via the manual controller 94.

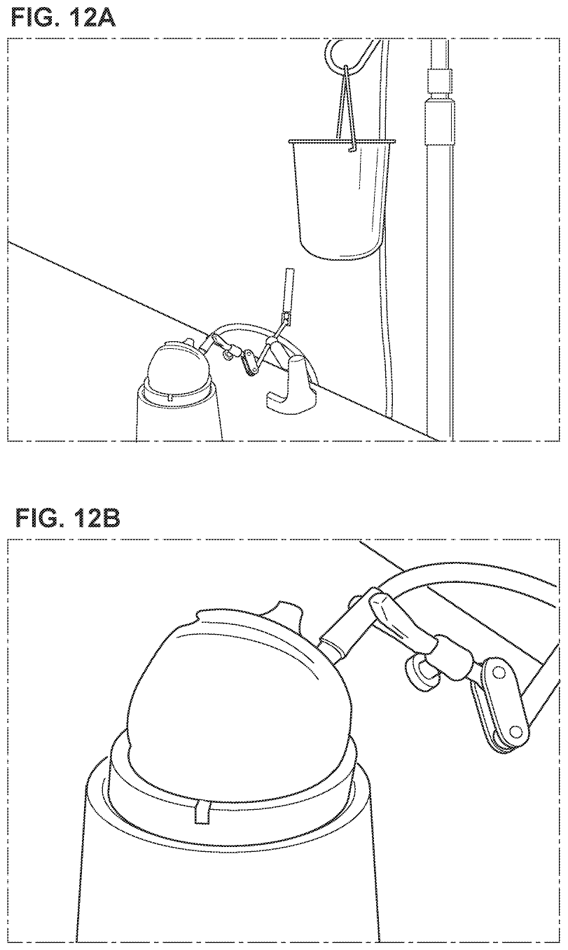

In accordance with the principles herein, a suitable training model can be provided to provide simulation training to surgeons. As discussed above, one exemplary training model is illustrated in FIGS. 5 and 6. In addition, another training model is illustrated in FIGS. 12A-12B, for example. The training model can provide an eye or other anatomical model that mimics the pressure, feel and tissue plasticity experienced during a particular surgical procedure. Training models can be formed using any suitable extrusion, printing or other fabrication process.

In addition, for the catheter 20A, 20B, 20C and catheter systems 40 described above, a catheter navigational component must pass force through the opposing longitudinal motion of the bifurcated layers 32. The relative translated force differential between the opposing layers 32 (when the sheath 34 is in place) results in pivoting of the catheter tip 22A, 22B, 22C. The ability to pass force is generally related to stiffness of the navigational component. Stiffness can be enhanced through material(s) selection (hardness/elasticity), geometry (thicker layers), or additional design elements (embedded prestressed elements, external sheath). Suitable materials for all inserted catheter system components include biocompatible polymers with tailorable material properties (e.g., polyurethane). Moreover, an overall length of catheter navigational component depends on ability to translate force.

A relationship exists between length and material stiffness approximated by cantilever with uniform and distributed load. While holding the catheter navigational component by proximal end, maximal displacement of the member (i.e., at the tip) is given as

$${δ}_{max} = \frac{q {L}^{4}}{8 EI} ,$$

where q is uniform load (in units Pascal/m), L is length (in units m), E is modulus of elasticity (in units Pascal), and I is moment of inertia (units of m⁴). Here,

$$I = \frac{1}{12} B {H}^{3} ,$$

where B is the long axis (in units m) and H is the short axis (in units m); q=pgBH, where ρ is density (in units of kg/m³), g is acceleration due to gravity (in units of m/s²).

In one example, q is a specific gravity of 1.15 for Smooth-On TASK 14 polyurethane; thus, the density=1150 kg/m³. In addition, the overall dimensions (l,h,b)=75×2×4 mm.

$$q = ρ gBH = 1150 * 9.8 * 0.004 * 0.002 = 0.09016 \frac{N}{m}$$

$$l : overall dimensions ( l , h , b ) = 75 \times 2 \times 4 mm$$

$$I = \frac{1}{12} {BH}^{3} = \frac{1}{12} 0.004 * 0.00 {2}^{3} = 2.6 \times {10}^{- 12} {m}^{4} \backslash$$

Further, it is desired to minimize deflection, so no more than 200% of H (0.002 m*2=0.004 m), material stiffness needs to be at least a minimum value, such as

$${δ}_{max} = \frac{q {L}^{4}}{8 EI} , where E = \frac{q {L}^{4}}{8 I {δ}_{max}}$$

In one example:

$$E = \frac{0.09016 * 0.07 {5}^{4}}{8 * 2.6 \times {10}^{- 12} * 0.004} = 3.4 \times {10}^{7} Pa , 34 MPa$$

In another example, Smooth-On TASK 14 polyurethane has E of 12.4 MPa/0.25=−50 MPa and qualified as a sufficient material.

In addition to the foregoing, an exemplary method of manufacturing a steerable, bidirectional catheter system may comprise the following steps: forming a catheter configured for insertion to a selected anatomical location; connecting the catheter to a suitable drive mechanism; and securing the catheter to the drive mechanism, if desired; attaching a sheath about the catheter, if desired; and forming portions or all of the system components of suitable disposable material.

Further, certain embodiments can be operated as follows, if desired: assemble device (sheath, collar, catheter, case); power on device, create incision (in model, animal, or human) to access target anatomical region, such as subretinal space, suprachoroidal space, or other anatomical region (or analog, in the case of model); insert the catheter tip, drive the catheter to insert sheathed catheter fully into suprachoroidal space (or analog); provide tip articulation as necessary to avoid obstacles/bunching against space-defining tissues; repeat drive and articulation as necessary to access desired location; remove collar from case; withdraw catheter and case while holding sheath still; use sheath as a port to access region and/or deliver therapeutics; and withdraw sheath.

Still further, it will be understood that the catheter 20A, 20B, 20C and catheter system 40 described above may also operate according to the following exemplary method. Specifically, a method of operating the catheter system 40 comprises providing the catheter 20A, 20B, 20C with the bifurcated shaft body 24 and the tip 22A, 22B, 22C coupled to the bifurcated shaft body 24, and the sheath 34 surrounding a portion of the bifurcated shaft body 24. The method further includes controlling selective deflection of the tip 22A, 22B, 22C of the catheter 20A, 20B, 20C by a navigation system 36 operatively coupled to the bifurcated shaft body 24 of the catheter 20A, 20B, 20C to form a delivery channel configured for access and/or a targeted delivery of a therapeutic payload. In some examples, controlling selective deflection of the tip 22A, 22B, 22C of the catheter 20A, 20B, 20C by a navigation system 36 coupled to the bifurcated shaft body 24 comprises translating a pair of opposing members 32 of the bifurcated shaft body 24 of the catheter 20A, 20B, 20C relative to each other via a drive mechanism, such that catheter flex would be refined to isolate motion of the tip 22A, 22B, 22C via the sheath 34, and the navigation system 36 controls articulation of the tip 22A, 22B, 22C. In another example, controlling selective deflection of the tip 22A, 22B, 22C of the catheter 20A, 20B, 20C by a navigation system 36 coupled to the bifurcated shaft body 24 comprises controlling selective deflection of the tip 22A, 22B, 22C of the catheter by an electromechanical mount 42 coupled to a drive system 44. Further, and in yet another example, controlling selective deflection of the tip 22A, 22B, 22C of the catheter 20A, 20B, 20C by the navigation system 36 coupled to the bifurcated shaft body 24 comprises controlling selective deflection of the tip 22A, 22B, 22C of the catheter 20A, 20B, 20C by the manual controller 94, and further comprising selectively inserting the catheter 20A, 20B, 20C into the manual controller 94, the manual controller 94 having at least one stationary element and at least one translating element.

The method may still further include delivering therapeutic payloads to a target location through the sheath 34, enabling an amount of therapeutic payloads to be delivered consistent with a size of an internal area of the sheath 34. The internal area of the sheath 34 is typically larger than an internal area of the catheter alone; as a result, a larger payload is able to be delivered by way of the sheath 34, for example. The method still further comprises providing access for a light source and/or other surgical device by the sheath 34, while minimizing stress to the adjacent tissues during operation. In another example, the method may include moving the tip 22A, 22B, 22C in a first direction or a second direction based on drive inputs deliverable via a mounting system to at least one of a first side and a second side of the catheter 20A, 20B, 20C. The method may still further comprise providing greater contact with a target by the first surface area 82a of the tip 22B of the catheter 20B than the second surface area 84a of the tip 22B of the catheter 20B, minimizing pressure against the target.

The method may also include bending the catheter 20A, 20B, 20C via the bifurcated shaft body 24 to produce two opposing members 32 connected to the tip 22A, 22B, 22C and shifting the opposing members 32 relative to each other, producing uniform, bidirectional or multi-directional flex of the tip 22A, 22B, 22C. The method also includes steering the tip 22A, 22B, 22C around one or more of a physiologic curvature or other obstacle, reducing damage to surrounding areas of a target area.

The method of operating the catheter 20A, 20B, 20C may further include passing force through opposing longitudinal motion of two opposing members 32 of the bifurcated shaft body via the navigation system 36, resulting in pivoting of the tip 22A, 22B, 22C due to relative translated force differential between the two opposing members 32 when the sheath 34 is in place. Still further, providing a catheter with a bifurcated shaft body and a tip coupled to the bifurcated shaft body, and a sheath surrounding a portion of the bifurcated shaft body may comprise providing the bifurcated shaft body 24 and the sheath 34 comprising biocompatible polymers having: (1) one or more of a hardness or an elasticity equal to a minimum value to minimize deflection; or (2) a geometry including a layer with an increased thickness.

In view of the foregoing, it will be understood that the device can be reused by replacing the catheter, collar and sheath with a new catheter, collar, and sheath. Alternatively, for disposable models the articulating device (case, collar, sheath, catheter, electromechanical mount) can all be discarded after use.

In addition, while several examples above describe the catheter 20A, 20B, 20C and catheter system 40 generally for use an ophthalmic catheter with areas and tissues in a human eye, the catheter 20A, 20B, 20C, catheter system 40 and all methods described above may alternatively and/or additionally also be used with any of following applications and still fall within (and have the many benefits of) the scope of the present disclosure. In particular, the catheter 20A, 20B, 20C, catheter system 40 and all methods may be used with retrobulbar applications, interventional radiology applications, intraperitoneal applications, and GI applications, including but not limited to applications adjacent to one or more of the pancreas, the duodenum, the intestinal tract, hepatic access, for example, and also splenic and peri-gastric applications. Further, the catheter 20A, 20B, 20C, catheter system 40 and all methods may also be used in retroperitoneal applications, such as providing access to GU tract (e.g., kidney, ureter, bladder), gynecologic areas (e.g., ovarian, uterine, adnexal applications), the spine, and tumors and/or disease. Still further, the catheter 20A, 20B, 20C, catheter system 40 and all methods may also be used with otolaryngology applications, such as sinus access, the parathyroid, esophageal/parathyroid, and head and neck tumors. The catheter 20A, 20B, 20C, catheter system 40 and all methods may still further be used in perivascular and orthopaedic applications, such as joint access, and neurological applications, including intracranial procedures and peripheral neurologic uses.

Moreover, it will also be understood that the systems and methods described above have several advantages. In particular, the steerable catheter 20A, 20B, 20C and the tips 22A, 22B, and 22C are navigable over an obstacle and/or restrictors in any tissue or area of the human body during use, while minimizing damage to the surrounding tissues and/or area. In addition, the novel asymmetrical tip designs enable the tips 22A, 22B and 22C to bend and flex during use, such that the tips 22A, 22B, 22C take the shape and form of a curved and/or a non-curved surface of portion of a tissue in which the tip 22A, 22B, 22C is contacting. Moreover, the sheath 34 coupled to the catheter 20A, 20B, 20C enables larger payloads of therapeutics to be delivered to a targeted area than previously used catheters, as described in detail above.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

As used herein, the terms “comprises,” “comprising,” “may include,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also may include the plural unless it is obvious that it is meant otherwise.

This detailed description is to be construed as examples and does not describe every possible embodiment, as describing every possible embodiment would be impractical, if not impossible. One could implement numerous alternate embodiments, using either current technology or technology developed after the filing date of this application.

Variations of the specific device configurations shown and described herein are within the scope of the principles of the present disclosure, and are included in all claims deriving therefrom.

1. A catheter comprising:
a bifurcated shaft body; and
a tip coupled to the bifurcated shaft body,
the tip configured to dissect a portion of a tissue in a controlled navigation for targeted delivery of a payload to an area of or within the tissue, and the tip adapted to bend along a shape of the area of the tissue.

2. The catheter of claim 1, wherein the tip is one or more of a bidirectional tip or a multi-directional tip, the tip further comprising one of a beveled tip or an asymmetric, double beveled tip, and further configured to provide access to regions of a human body for surgical and/or therapeutic interventions.

3. The catheter of claim 1, wherein the tip is a double beveled tip, the double beveled tip having a first side with a first surface area and a second side with a second surface area, the first surface area greater than the second surface area of the double beveled tip, such that the first surface area has a contact area greater than a contact area of the second surface area during operation of the catheter, the first and second surface areas are adapted to contact tissues.

4. The catheter of claim 1, wherein the bifurcated shaft body includes a proximal end, a distal end and a slit extending along a length of the body from a position near the distal end and through the proximal end, such that the proximal end includes a pair of opposing members coupled to the tip at the distal end.

5. The catheter of claim 1, wherein the bifurcated shaft body includes pair of opposing layers, the opposing layers forming sliding members adapted to shift relative to each other, producing a uniform flex along the length of the shaft body.

6. The catheter of claim 1, wherein a portion of the catheter is adapted to be disposed within a sheath, and the tip is adapted to move separate from the bifurcated shaft body via the sheath and in a direction about a longitudinal axis of the bifurcated shaft body while the bifurcated shaft body uniformly flexes relative to the longitudinal axis of the bifurcated shaft body.

7. The catheter of claim 3, wherein the first side of the double beveled tip has a first length and the second side of the double beveled tip has a second length, the first length longer than the second length.

8. The catheter of claim 1, wherein the tip includes variable aspect ratios, imparting minimal stress during operation.

9. The catheter of claim 1, wherein the catheter is used for accessing regions of an eye for surgical and/or therapeutic interventions, and the tip is adapted to minimize injury to regions of the eye, including the suprachoroidal space (SCS).

10. The catheter of claim 1, wherein a portion of the catheter is adapted to be disposed within a sheath, such that the tip outwardly extends from an end of the sheath and the bifurcated shaft body is disposed within the sheath, one or more of reducing flex of the bifurcated shaft body, isolating movement of the tip, and providing access and/or targeted delivery of a therapeutic payload to an area of tissue via the sheath.

11. A catheter system comprising:
a catheter having a bifurcated shaft body and a tip coupled to the bifurcated shaft body;
a sheath surrounding a portion of the bifurcated shaft body, the tip extending outwardly from an end of the sheath; and
a navigation system operatively coupled to the bifurcated shaft body of the catheter for controlling selective deflection of the tip of the catheter,
where the sheath is adapted to form a delivery channel to an area of tissue, the delivery channel configured for access and/or targeted delivery of a therapeutic payload to the area of tissue.

12. The system of claim 11, wherein the navigation system is an electromechanical mount operatively coupled to the catheter, the electromechanical mount configured to translate a pair of opposing members of the bifurcated shaft body of the catheter relative to each other via a drive mechanism, such that catheter flex would be refined to isolate motion of the tip via the sheath, and the electromechanical mount controls articulation of the tip.

13. The system of claim 12, wherein the electromechanical mount houses a motor and a processor configured to receive inputs from a drive system, the electromechanical mount configured to translate sliding layers of the catheter relative to each other, the mount operably connectable to the catheter and to the drive system, wherein the sheath and the drive system, via the electromechanical mount, are configurable to control directional movement of the tip of the catheter to steer the tip, and the electromechanical mount includes an enclosure having an opening with a collar, and the sheath is coupled to the collar.

14. The system of claim 12, wherein the electromechanical mount is coupled to a control box and a drive system for controlling the movement of the catheter assembly.

15. The system of claim 11, wherein the navigation system comprises a manual controller, and the catheter is selectively inserted into the manual controller, the manual controller having at least one stationary element and at least one translating element, the at least one translating element adapted to be controlled by a user.

16. The system of claim 11, wherein the tip is a bidrectional tip, the tip further comprising one of a beveled tip or an asymmetric, double beveled tip, and further configured to provide access to regions of a human body for surgical and/or therapeutic interventions.

17. The system of claim 16, the double beveled tip having a first side with a first surface area and a second side with a second surface area, the first surface area greater than the second surface area of the double beveled tip, such that the first surface area has a greater contact area than the second surface area, the first and second surface areas are adapted to contact tissues.

18. The system of claim 11, wherein the tip includes various aspect ratios, imparting minimal stress.

19. The system of claim 11, wherein the catheter is configured to access regions of an eye for surgical and/or therapeutic interventions and the tip is adapted to minimize injury to regions of the eye, including the suprachoroidal space (SCS).

20. The system of claim 11, wherein the sheath includes an internal area for one or more of: (1) receiving therapeutic payloads and enabling delivery of therapeutic payloads consistent in size with the internal area of the sheath; or (2) providing access for a light source and/or other surgical device, after removal of the catheter from the sheath

21. The system of claim 11, the catheter further comprising a first side and a second side, wherein the tip moves in a first direction or a second direction based on drive inputs deliverable via an electromechanical mount to at least one of the first side and the second side of the catheter.

22. The system of claim 11, the catheter having a width greater than height to provide access or deliver payload toward posterior regions of an eye, wherein the larger width to height ratio allows for efficient displacement of tissue, thereby increasing the delivery channel payload while minimizing tissue displacement.

23. The system of claim 11, the system configured to selectively form a pathway to a target location, and wherein the therapeutic payloads are delivered to the target location through one or more of the pathway or the sheath directly, or by guiding a syringe or other delivery device through one or more of the pathway or the sheath.

24. The system of claim 11, further comprising one or more of a hand-driven apparatus, a mechanical drive system, or a motor-driven drive system for steering the bidirectional tip of the catheter.

25. The system of claim 11, wherein the sheath is one or more of semi-rigid, disposable, and configured to selectively constrain catheter flex of the portion of the bifurcated body of the catheter disposed within the sheath.

26. A method of operating a catheter system, the method comprising:
providing a catheter with a bifurcated shaft body and a tip coupled to the bifurcated shaft body, and a sheath surrounding a portion of the bifurcated shaft body; and
controlling selective deflection of the tip of the catheter by a navigation system operatively coupled to the bifurcated shaft body of the catheter to form a delivery channel configured for access and/or a targeted delivery of a therapeutic payload.

27. The method of claim 26, wherein controlling selective deflection of the tip of the catheter by a navigation system coupled to the bifurcated shaft body comprises translating a pair of opposing members of the bifurcated shaft body of the catheter relative to each other via a drive mechanism, such that catheter flex would be refined to isolate motion of the tip via the sheath, and the navigation system controls articulation of the tip.

28. The method of claim 26, wherein controlling selective deflection of the tip of the catheter by a navigation system coupled to the bifurcated shaft body comprises controlling selective deflection of the tip of the catheter by an electromechanical mount coupled to a drive system.

29. The method of claim 26, wherein controlling selective deflection of the tip of the catheter by a navigation system coupled to the bifurcated shaft body comprises controlling selective deflection of the tip of the catheter by a manual controller, and further comprising selectively inserting the catheter into the manual controller, the manual controller having at least one stationary element and at least one translating element.

30. The method of claim 26, further comprising delivering therapeutic payloads to a target location through the sheath, enabling an amount of therapeutic payloads to be delivered consistent with a size of an internal area of the sheath.

31. The method of claim 26 further comprising providing access for a light source and/or other surgical device by the sheath, while minimizing stress to the adjacent tissues during operation.

32. The method of claim 26, further comprising moving the tip in a first direction or a second direction based on drive inputs deliverable via a mounting system to at least one of a first side and a second side of the catheter.

33. The method of claim 26, further comprising providing greater contact with a target by a first surface area of the tip of the catheter than a second surface area of the tip of the catheter, minimizing pressure against the target.

34. The method of claim 26, further comprising bending the catheter via the bifurcated shaft body to produce two opposing members connected to the tip and shifting the opposing members relative to each other, producing uniform, bidirectional or multi-directional flex of the tip.

35. The method of claim 26, further comprising steering the tip around one or more of a physiologic curvature or other obstacle, reducing damage to surrounding areas of a target area.

36. The method of claim 26, further comprising passing force through opposing longitudinal motion of two opposing members of the bifurcated shaft body via the navigation system, resulting in pivoting of the tip due to relative translated force differential between the two opposing members when the sheath is in place.

37. The method of claim 26, wherein providing a catheter with a bifurcated shaft body and a tip coupled to the bifurcated shaft body, and a sheath surrounding a portion of the bifurcated shaft body comprises providing the bifurcated shaft body and the sheath comprising biocompatible polymers having: (1) one or more of a hardness or an elasticity equal to a minimum value to minimize deflection; or (2) a geometry including a layer with an increased thickness.

38. A catheter tip system comprising an asymmetric double bevel tip adapted to be coupled to a shaft of a catheter, the tip adapted to provide greater tip contact area preferentially with one tissue, permitting minimizing of pressure preferentially during a surgical procedure.

39. The system of claim 38, further comprising one or more of a hand-driven apparatus, a mechanical drive system, or a motor-driven drive system, each of the hand-driven apparatus, the mechanical drive system, and the motor-driven drive system for steering the tip.

40. The system of claim 38, further comprising a bifurcated shaft coupled to the tip, and a sheath for receiving a portion of the bifurcated shaft, such that the tip outwardly extends from an end of the sheath and the bifurcated shaft body is disposed within the sheath, one or more of reducing flex of the bifurcated shaft body, isolating movement of the tip, and providing access and/or targeted delivery of a therapeutic payload to an area of tissue via the sheath.

41. The system of claim 38, wherein one or more of the bifurcated shaft body and the sheath comprises at least one biocompatible polymer having one or more of: (1) a hardness or an elasticity equal to a minimum value to minimize deflection; or (2) a geometry including a layer with an increased thickness.

42. The catheter of claim 1, wherein the regions of a human body for surgical and/or therapeutic interventions include any one or more of ophthalmologic, retrobulbar, gastrointestinal, retroperitoneal, otolaryngologic, perivascular, orthopaedic or neurologic regions.

43. The system of claim 11, wherein the area of tissue includes any one or more of ophthalmologic, retrobulbar, gastrointestinal, retroperitoneal, otolaryngologic, perivascular, orthopaedic or neurologic areas in the human body.
